SEQUENCE LISTING (4 sequences)

SEQ ID NO: 1 - protein, 20 aa, Artificial Sequence
  synthetic peptide
VSHDLRTPLTRIRLATEMMS

SEQ ID NO: 2 - protein, 16 aa, Artificial Sequence
  synthetic peptide
HDLRTPLARIRRATEM

SEQ ID NO: 3 - protein, 22 aa, Artificial Sequence
  synthetic peptide
ASDVSHDLRTPLTRRRPVNMMS

SEQ ID NO: 4 - protein, 24 aa, Artificial Sequence
  synthetic peptide
ASDVSHDYVVALRTPLTRRRPVQQ

(19) United States
(12) Patent Application Publication
(10) Pub. No.: US 2004/0015298 A1
(43) Pub. Date: Jan. 22, 2004

(54) Multiple sequence alignment

(76) Inventors: Mark Swindells, Lincs (GB); Mark Rae, London (GB)

Correspondence Address: DARBY & DARBY P.C., P. O. BOX 5257, NEW YORK, NY, 10150-5257, US

(21) Appl. No.: 10/221,833

(22) PCT Filed: Mar. 14, 2001
(86) PCT No.: PCT/GB01/01110

(51) Int. Cl. G06F 19/00; G01N 33/48; G01N 33/50

(52) U.S. Cl. 702/19; 702/20

(57) ABSTRACT

The invention relates to a method of aligning a plurality of sequences. In a similar way to known multiple alignment methods, the method of the invention uses a profile for the nominated sequence in an alignment strategy. The key novel concept behind the method of the invention is to allow the profile to be extended in regions where gaps are desired. This alternative strategy is implemented using pre-generated profiles as a basis for the multiple alignment.

The invention relates to a method of aligning a plurality of sequences.

A high quality multiple alignment of nucleotide or protein sequences is one where the total evolutionary distance is minimised over the entire set of sequences. To achieve this, gaps must be progressively inserted into the alignment as each additional sequence is added to the alignment. However, in the interests of producing an alignment that corresponds with our knowledge of where insertions/deletions typically occur between homologous protein structures, while at the same time being both aesthetically pleasing and easy to interpret, the number of gaps inserted should be no more than is necessary to maintain correctly-equivalenced residues, with gapped regions from homologous proteins lining up wherever this is possible.

Standard multiple alignment tools (such as Clustal W; Thompson et al., (1994) 22(22): 4673-4680) use a number of steps in order to form an alignment. Assuming that the sequences of interest have already been identified by a database search, the first step is usually to calculate all pairwise similarities in order to establish which sequences are most similar to each other. Then, using these similarities, the multiple alignment is constructed in a stepwise manner utilising either two sequences or aligned sets of sequences. A diagrammatic tree showing these relationships is presented in FIG. 1.

In the case illustrated in FIG. 1, the order would be; A with B, C with D, (AB) with (CD).

Overall, this approach can be extremely time consuming. For an alignment containing N sequences, there would need to be (N×[N−1]) initial comparisons followed by another [N−1] alignments to generate the multiple alignment from the tree.

For each position in the alignment, the average score between all pairs of sequences in the aligned sets are used to calculate the average score for that position. Thus, for an alignment between previously aligned sets of 2 and 4 sequences, each position will require 8 comparisons.

Usually, multiple alignment approaches give no consideration to where gaps have been previously inserted, rather relying on the overall similarity between the sequences.

However, there are also more advanced methods that allow the gap penalties to be varied on this basis. For instance, in the Clustal W alignment program, it is possible to have the gap opening penalty decreased by a third in areas where gaps already exist. Other ways of altering gap penalties are based on features such as the overall similarity of the sequences, sequence length and differences in sequence length.

Many of the latest database search methods achieve additional sensitivity by using the sequences identified in a standard database search (such as blast) to construct a profile of position specific residue preferences that more accurately describe the key features of a homologous family in question. By continually refining the profile after each search has been completed, these methods have the opportunity to identify yet more relationships, though after about ten such iterations, most searches will have converged.

The point for multiple alignment is that these profiles already contain valuable information about how each of the detected sequences compares against the query profile. Unfortunately, while the standard database search procedures that produce these profiles are extremely sensitive, they perform their comparisons like a standard pairwise search and have no additional technology to produce a high quality multiple alignment at the end.

Traditional multiple alignment methods take a considerable time to generate alignments for any more than three sequences. It is also true to say that even the approaches described above are an approximation because there is no guarantee that the alignment that is globally the best has been made by fixing the alignments of more similar sequences early on and progressively aligning the more distant sets of relatives.

There is thus a great need for an improved method of aligning multiple sequences that does not suffer from these disadvantages.

### SUMMARY OF THE INVENTION

According to the invention, there is provided a computer-implemented method of aligning a plurality of protein or nucleic acid sequences comprising the steps of:

a) performing an alignment of a query sequence to a target sequence using a dynamic programming algorithm that constructs the alignment using a scoring matrix profile to provide an alignment score for aligning amino acid residues together, wherein suitable candidate residues for alignment are given a positive score and unsuitable candidate residues are given a negative score, and negative score penalties are generated both for opening and for extending a gap in one of the sequences in the alignment; and

b) repeating step a) for each sequence to be aligned;

wherein the scoring matrix profile may be modified after each alignment step a) and before being used to generate the alignment of the next sequence, and wherein if the best scoring alignment requires that a gap be introduced into the profile, the profile is modified by inserting the residues from the query sequence that match up with the gap region.

In a similar way to known multiple alignment methods, the method of the invention uses a profile for the nominated sequence in an alignment strategy. The key novel concept behind the method of the invention is to allow the profile to be extended in regions where gaps are desired. Using pre-generated profiles as a basis for the multiple alignment permits this alternative strategy to be implemented. Preferably, a pairwise alignment strategy is used.

By “target sequence” is meant the nominated sequence on which the multiple alignment strategy is to be based. It is this sequence which is represented in the profile when the multiple alignment is commenced. This profile for this nominated target sequence is then aligned against a plurality of query sequences in turn, with the profile being modified by the alignment algorithm as the alignment proceeds.

In theory, any number of query sequences may be aligned against the profile for the target sequence. However, preferably, a selection of related sequences are used. Such a selection may be selected from the results of an iterative alignment program such as PSI-BLAST.

Preferably, the method of the invention is used to perform multiple alignments of protein sequences. Accordingly, the more detailed aspects of the invention that are described below refer to only to amino acid residues, in the context of aligning protein sequences. However, the skilled reader will appreciate that the method of the invention is equally applicable to the alignment of nucleic acid molecules. Furthermore, it is envisaged that this method could easily be extended to allow the alignment of any string of letters where individual letter types have defined degrees of similarity. By “letter” is meant any character forming strings which it is desired to align together, and thus “letter” may include an ascii code.

In a preferred embodiment of the invention, the query sequences are aligned against the target sequence in order of their similarity to the target sequence. This degree of similarity may be assessed by degree of evolutionary divergence, for example, as defined by a similarity score generated by an alignment program such as PSI-BLAST. Preferably, a threshold similarity score is used to define the limit of similarity that a query sequence may display with a target sequence in order to be included in the multiple alignment method. This prevents the program that implements the process of the invention from attempting to align sequences that are too dissimilar to align to the target sequence. For example, for a sensible alignment to be generated, attempting to align a sequence that was not detected as being related to the target sequence by PSI-BLAST (and hence in this example the profile to be used in the alignment) would be inadvisable.

The basis of the novel algorithm that implements the method of the invention is the global alignment of two sequences using a dynamic programming algorithm, such as the pairwise alignment strategy described by Myers & Miller (Myers and Miller, Comput Appl Biosci (1988) 4(1):11). However, the novel method uses a profile-based scoring scheme when constructing the alignment. This is where the score for aligning two residues or nucleotides is not fixed globally, but varies with position along one of the sequences, this sequence always being the nominated sequence for which the multiple alignment will be constructed.

This profile is then used to generate the alignment with a target sequence. However, one or the key points for generating a multiple sequence alignment using this approach is to allow further modification of the profile. After each pairwise alignment is calculated, the profile is modified as shown in FIG. 2, as each of the sequences is aligned against it. Where the alignment calls for a gap in the profile, the profile is modified by inserting, from the aligned sequence, the residues or nucleotides that match up with the gap. These inserted residues or nucleotides are marked as such, as they have an effect on subsequent alignments of query sequences. The scoring values that these inserted residues are given may be taken from a standard scoring matrix such as any of the BLOSUM or point accepted mutation (PAM) series. A particularly suitable matrix has been found to be the widely used BLOSUM-62 matrix. Other suitable matrices will be clear to those of skill in the art.

After the pairwise alignment of each target sequence with the query sequence, the profile for the target sequence is modified before being used to produce the alignment for the next query sequence. Areas in the profile that have been modified are marked as such, as they affect the way that the alignment is scored in the dynamic programming step. This procedure is repeated for each sequence in turn until the complete alignment is produced.

In a preferred embodiment of the invention, if amino acid residues in a second or subsequent query sequence are aligned against a modified region of the profile where residues have been inserted and said amino acid residues are assigned a negative score, their score is reset to zero, such that multiple sequences that have similar regions that were not present in the original profile may be aligned together without penalty while at the same time allowing the alignment score to be increased for correctly aligned regions that have a positive score.

If the alignment of a second or subsequent query sequence requires that a gap be inserted or extended into the sequence that is being aligned against the profile and this gap falls within a modified region of the profile where residues have been inserted, no negative score penalty is generated. In this fashion, a sequence that would normally align against the profile without the need for a gap can be aligned without an inserted region interfering with the alignment.

The scoring matrix profile used in the alignment method may be a profile generated by running a profile-based alignment algorithm such as PSI-BLAST on the target sequence. However, a default scoring matrix may be used, if necessary. Suitable scoring matrices will be well known to those of skill in the art and include the BLOSUM and PAM matrices, particularly PAM 250 and BLOSUM 62. Preferably, the profile originates from running PSI-BLAST with the target sequence.

If a query sequence has previously been aligned by another method, and it has been discovered that the query sequence can align against the nominated target sequence in multiple locations, it is necessary to put this sequence through the algorithm multiple times, one for each of these ‘local hits’. The alignment produced for each appearance of the sequence must be constrained so that the correct local hit is chosen, rather than aligning the best area repeatedly. This constraint mechanism can also be used to make sure that particular areas of interest that have been previously identified are preserved by the alignment procedure.

Accordingly, this aspect of the method provides that if a query sequence is known to align against a target sequence in multiple locations such that multiple alignment hits are generated by the alignment of these sequences, then step a) is repeated for each location at which the sequences align, and for each separate iteration, the alignment of the sequences is constrained to one particular alignment location. This mechanism of constraint excludes regions from consideration by the dynamic programming algorithm by setting the matrix profile scores in the excluded region to a large negative value that is far more negative than any value that would occur naturally during the execution of the algorithm. Conveniently, this large negative value that is assigned is the largest negative value that can be stored by the computer on which the alignment method is being performed.

The effect of using a constraint mechanism as described above can be seen from FIG. 3. In this figure, the calculated alignment enters and exits the constrained region in the centre at the given points at either comer. However, within the central region, and the two other areas at either side, the alignment algorithm is free to proceed as normal. This means that it is possible approximately to specify a general area of interest and the alignment will find the best alignment within that region.

One advantage of this algorithm is that it can be performed in O(n) time, where a full multiple alignment requires O(n²) time. This means that the primary use of the method of the present invention is in interactive systems, where the alignments must be produced quickly in response to user requests. In such situations, it is expected that the sequences that are required to be aligned will have already been shown to have a reasonable degree of similarity, at least within certain regions, which is where this method performs best.

As can be seen from the simple example given in FIG. 4, the differences between this algorithm and a full multiple alignment are minor. However these differences grow as the sequences that are required to be aligned begin to increase in difference.

According to a further aspect of the invention, there is provided a computer apparatus adapted to perform a method according to any one of the aspects of the invention described above.

In a preferred embodiment of the invention, said computer apparatus may comprise a processor means incorporating a memory means adapted for storing data relating to amino acid or nucleotide sequences; means for inputting data relating to a plurality of protein or nucleic acid sequences; and computer software means stored in said computer memory that is adapted to align said plurality of protein or nucleic acid sequences and output a multiple alignment of said sequences.

The invention also provides a computer-based system for aligning a plurality of protein or nucleic acid sequences comprising means for inputting data relating to a plurality of protein or nucleic acid sequences; means adapted to align said plurality of protein or nucleic acid sequences; and means for outputting a multiple alignment of said sequences.

The system of this aspect of the invention may comprise a central processing unit; an input device for inputting requests; an output device; a memory; and at least one bus connecting the central processing unit, the memory, the input device and the output device. The memory should store a module that is configured so that upon receiving a request to align a plurality of protein or nucleic acid sequences, it performs the steps listed in any one of the methods of the invention described above.

In the apparatus and systems of these embodiments of the invention, data may be input by downloading the sequence data from a local site such as a memory or disk drive, or alternatively from a remote site accessed over a network such as the internet. The sequences may be input by keyboard, if required.

The generated alignment may be output in any convenient format, for example, to a printer, a word processing program, a graphics viewing program or to a screen display device. Other convenient formats will be apparent to the skilled reader.

The means adapted to align said plurality of protein or nucleic acid sequences will preferably comprise computer software means, such as the computer software discussed in more detail below. As the skilled reader will appreciate, once the novel and inventive teaching of the invention is appreciated, any number of different computer software means may be designed to implement this teaching.

According to a still further aspect of the invention, there is provided a computer program product for use in conjunction with a computer, said computer program comprising a computer readable storage medium and a computer program mechanism embedded therein, the computer program mechanism comprising a module that is configured so that upon receiving a request to align a plurality of protein or nucleic acid sequences, it performs the steps listed in any one of the methods of the invention described above.

The invention will now be described by way of example with particular reference to a specific algorithm that implements the process of the invention. As the skilled reader will appreciate, variations from this specific illustrated embodiment are of course possible without departing from the scope of the invention.

### BRIEF DESCRIPTION OF THE FIGURES

FIG. 1 shows the evolutionary relationships between protein sequences as a phylogenetic tree.

FIG. 2 illustrates the way by which the profile of the nominated target sequence is modified by the insertion of a gapped region.

FIG. 3 illustrates the effect of the constraints imposed on alignments that have excluded regions specified.

FIG. 4 shows an alignment generated by the process of the invention. The individual alignments were produced using a standard Myers-Miller global alignment algorithm, whilst the multiple alignment was produced using Clustal W.

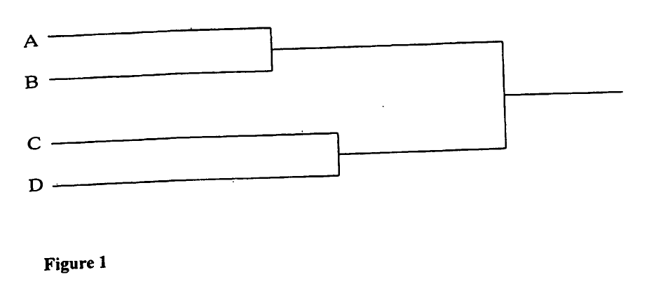

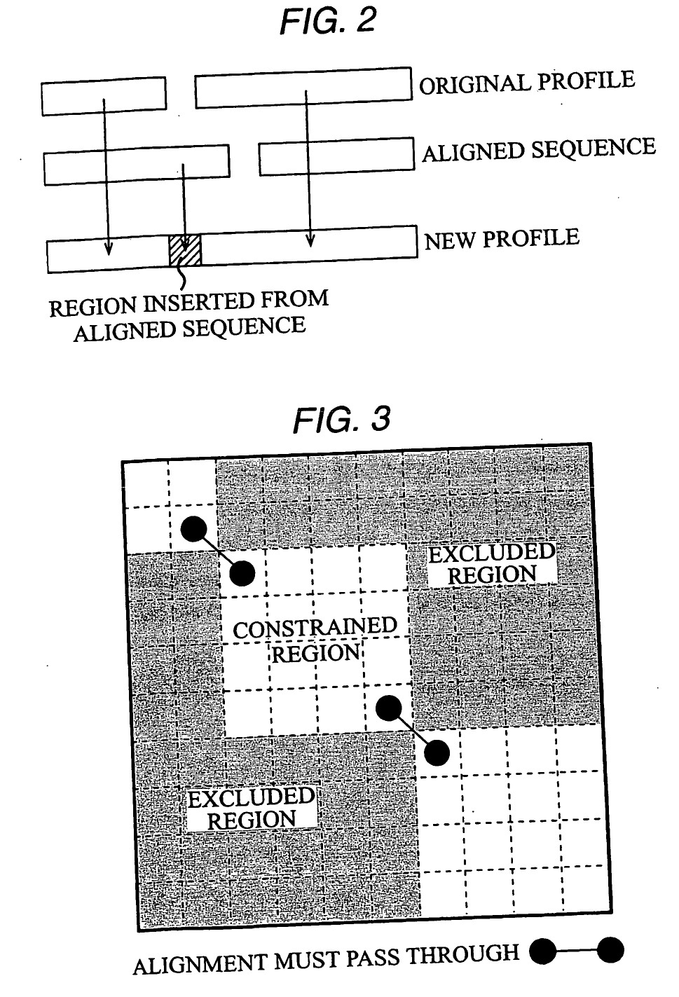

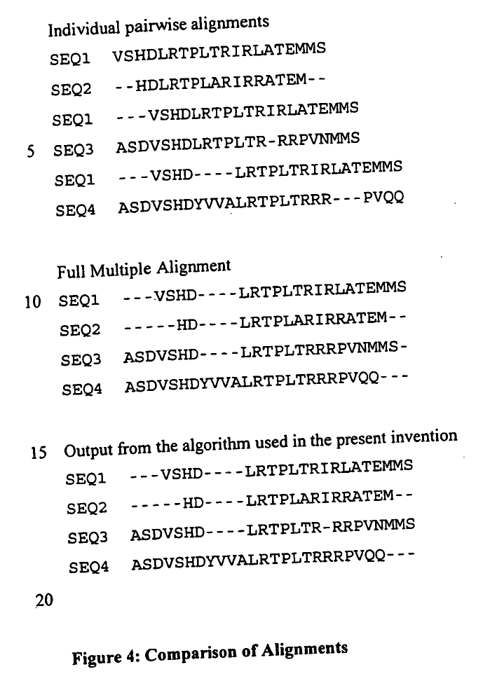

### EXAMPLES

### 1. Definitions

1.1 Sequences

Let L be an member of the alphabet R, which consists of all of the valid amino-acid (residue) types.

Then a protein sequence S consists of a series of letters Lᵢ, where i=1 . . . N and N is the length of the sequence.

S=L_{i=1 . . . N}:LᵢεR (1)

1.2 PAM Matrices

PAM matrices consist of a set of log-probability scores, M_{i,j}, i, j ε R, for the mutation of one letter Lᵢ into another Lⱼ in two evolutionary related sequences.

1.3 Profiles

A profile P is similar to a PAM matrix, except rather than having a fixed value for each i, j pair, the probability scores for a residue mutating into another is different for each residue L in the corresponding sequence S.

P_{i,j}=M′_{L}_{ᵢ}_{,j}:i=1 . . . N,jεR (2)

where M′ is a position specific mutation probability.

### 2. Sequence Alignment

2.1 Description of Problem

The alignment, A_{k,1}, of a set sequences Sₗ:l=1 . . . n is the arrangement of all or some of the residues in the sequences such that the summing of all of the mutation scores M is maximised.

That is to say, the values of A_{k,1}:l=1 . . . n are the positions in the sequences Sₗ which are all aligned together.

The alignment is subject to the following constraint, where a is the length of the alignment, which does not necessarily cover the whole range of all of the sequences.

A_{k+1,j}>A_{k,j}:∀lε{1 . . . n},k=1 . . . (a−1) (3)

This constraint means that the sequences cannot ‘loop back’ on themselves to produce an alignment, however ‘gaps’ can be inserted in the alignment. The insertion of these gaps may be subject to a penalty, which is subtracted from the score obtained by the summing of the M values.

2.2 Pairwise Alignment

The calculation of the best multiple alignment for more than a few sequences at a time is computationally expensive, therefore normally only pairwise alignments are calculated, that is alignments involving only two sequences.

The standard algorithms for producing a pairwise alignment are all based on the principle of dynamic programming. The individual algorithms are all variations involving differing constraints on the calculations, such as Smith-Waterman which does not allow scores to go negative.

2.2.1 Dynamic Programming

If we wish to align two sequences S and S′ of lengths N and N′ respectively, then we construct a score matrix T_{m,n} and calculate its elements as follows.

D=T_{m-1,n-1}+M_{L}_{ₘ}_{,L′}_{ₙ} (4)

or if we are using a profile for sequence S

D=T_{m-1,n-1}+P_{m,L′}_{ₙ} (5)

G1=T_{g,n-1}+P_{m,L′}_{ₙ}+G(m−g−1):gε{1 . . . m−2} (6)

G2=T_{m-1,g}+P_{m,L′}_{ₙ}+G(n−g−1):gε{1 . . . n−2} (7)

where G(p) is the penalty for inserting a gap of length p

T_{m,n}=max(D, G1, G2) (8)

The values of T_{m,n} obviously must be calculated with m and n strictly increasing.

Once the matrix T has been calculated the alignment is produced by tracing back through the matrix from a given starting point, the way the alignment goes through the matrix depending on the value chosen in equation 8. The starting point for this procedure also depends on the various variations of the algorithm.

2.2.2 Gap Penalty

The gap penalty G(p) used in the dynamic programming algorithm is used to reflect the idea that having to insert gaps into an alignment is not desirable, and is therefore always negative. The exact form and values of the penalty depends on the variation of the algorithm being used and the scoring matrix m which is being used. However the most commonly used penalty is of the form.

G(p)=G₀+Gₑ.p:G₀<0,Gₑ≦0 (9)

where G₀ is the initial penalty for opening a gap, and Gₑ is the incremental penalty for extending the gap.

### 3. Fast Multiple Alignment

The following section describes another variation on the dynamic programming algorithm which allows multiple sequences to be aligned by performing a series of n−1 pairwise alignments.

3.1 Profile Modification

This algorithm uses one reference sequence as the basis for the alignment, and it requires that a profile exist for this sequences. If one is not available a default one is easily generated from a suitable PAM matrix. P_{i,j}=M_{L}_{₁}_{,j}

Each sequence S₁:i=2 . . . n is aligned in turn against the profile P corresponding to sequence S₁ to produce an alignment A.

If the alignment requires that any gaps be inserted into the reference sequence, that is ∃kε{1 . . . a}:A_{k+1,2}>A_{k,2}+1 then a new profile, P′ is generated as follows.

z=A_{k+1,2}−A_{k,2}−1 (10)

P′_{i,j}=P_{i,j}:i=1 . . . A_{k,1},∀jεR (11)

P′_{A}_{_{k,1}}_{+i,j}=M_{L′}_{_{Ki-z}}ⱼ:i=1 . . . z,∀jεR (12)

P′_{i,j}=P_{i-Z,j}:i=A_{k+1,1}+z . . . a+z,∀jεR (13)

This new profile is then used for each subsequent pairwise alignment.

3.2 Gaps

Whenever a gap is inserted into a profile it is recorded as such, denoted by Iᵢ=1 if Pᵢ was inserted using the above procedure. This is then used to modify the behaviour of equations 5-7.

The first modification is mismatches, that is negatively scoring residue pairs are ignored if they are within a gap region. So equation 5 becomes

$$D = \{ \begin{matrix}{T}_{m - 1 , n - 1} + max ( {P}_{m , {{L}^{′}}_{n}} , 0 ) \\ {T}_{m - 1 , n - 1} + {P}_{m , {{L}^{′}}_{n}}\end{matrix} {I}_{m} = 1 otherwise \qquad ( 14 )$$

Secondly, if the alignment being calculated requires the insertion of a gap, and this new gap overlaps or is adjacent to one of the profile insertions, then the gap penalty is only the amount required to extend the gap from the size of the insertion up to the required size. So equation 6 becomes

G1=T._{g,n-1}+P_{m,L′}_{ₙ}+G(m−g−1)−G(e):gε{1 . . . m−2} (15)

Where G(e) is the cost that is associated with the inserted gap. That is, e is the number of Iₘ=1 residues within the new gap.

Equation 7 is modified similarly.

G2=T_{m-1,g}+P_{m,L′}_{ₙ}+G(n−g−1)−G(e):gε{1 . . . n−2} (16)

3.2 Constraining Alignments

When generating a profile from iterative sequence comparison methods, relationships between sequences are also generated, and these known relationships may identify regions of similarly between sequences which are required to be preserved by the alignment procedure. This can be accomplished by modifying the generation of the score matrix T to ensure that the generated alignment passes through these regions. So if we are aligning sequences S and S′ and we know that region a . . . b:1≦a<b≦N and a′ . . . b′:1≦a′≦b′≦N′ should be aligned then the generation of the score matrix equation 8 can be modified as follows

$${T}_{m , n} = \{ \begin{matrix}max ( D , G1 , G2 ) & a \leq m \leq b , {a}^{′} \leq n \leq {b}^{′} \\ max ( D , G1 , G2 ) & m < a , n < {a}^{′} \\ max ( D , G1 , G2 ) & m > b , n > {b}^{′} \\ MINVALUE & otherwise\end{matrix} \qquad ( 17 )$$

where MINVALUE is a highly negative number which would discount it from ever being considered as part of an alignment, usually the most negative number capable of being represented.

### 4. Examples

The following shows profile modification sequence from section 3.1

4.1 Profile Modification

| | |
|---|---|
| integer N1 | = length of sequence 1 / original profile |
| integer N2 | = length of sequence 2 |
| integer R | = number of letters |
| integer GL | = length of gap |
| integer G1 | = gap position in sequence 1 |
| integer G2 | = gap position in sequence 2 |

| | |
|---|---|
| integer S2(N2) | # Second Sequence |
| integer P1(N,R) | # Original profile |
| integer P2(N+GL,R) | # New Profile |
| integer M(R,R) | # PAM matrix |
| for i = 1 to G1−1 | |

for j = 1 to R

P2(i,j) = P1(i,j)

endfor

endfor
for i = 0 to GL−1

for j = 1 to R

P2(G1+i,j) = M(S2(G2+i),j)

endfor

endfor
for i = G1 to N1

for j = 1 to R

P2(i+GL,j) = P1(i,j)

endfor

endfor

4.2 Alignment

This shows an example of the modified dynamic programming algorithm shown in section 3.2. This example also keeps a running score of the best places to insert gaps, rather than searching explicitly for them each time, as implied by equations 6, 7, 15, 16.

| | |
|---|---|
| integer N1 | = length of sequence/profile 1 |
| integer N2 | = length of sequence 2 |
| integer GO | = gap opening penalty |
| integer GE | = gap extension penalty |

| | |
|---|---|
| integer S2(N2) | # Second Sequence |
| integer T(N1,N2) | # Score matrix |
| integer P(N1,R) | # Profile |
| integer G(N1) | # Profile insertions |
| integer hscore | # Holds best ‘horizontal’ gap jump score |
| integer vscore(N2) | # Holds best ‘vertical’ gap jump score |

# Initialise boundary conditions
for i = 1 to N1

T(i, 1) = P(i, S2(1) ) ;

endfor
for j = 1 to N2

T(1, j) = P(1, S2(j) ) ;
if G(1) = 1

vscore(j) = T(1,j)

else

vscore(j) = T(1,j)+GO−GE

endif

endfor
# Perform calculations
for i = 2 to N1

hscore = T(i,1)+GO−GE
for j = 2 to N2

sc = P(i,S2(j) )
if G(i) = 1

if sc < 0

sc = 0

endif

endif
maxscore = T(i−1, j−1) + sc;
score = sc + hscore;
if score > maxscore

maxscore = score

endif
score = sc + vscore(j) ;
if score > maxscore

maxscore = score

endif
T(i,j) = maxscore
hscore = hscore + GE
if T(i−1,j)+GO−GE > hscore

hscore = T(i−1,j)+GO−GE

endif
if G(i) <> 1

vscore(j) = vscore(j) + GE

endif
if G(i) = 1 or G(i+1) = 1

if T(i,j−1) > vscore(j)

vscore(j) = T(i,j−1)

endif

else

if T(i,j−1)+GO−GE > vscore(j)

vscore(j) = T(i,j−1)+GO−GE

endif

endif

endfor

endfor

1. A computer-implemented method of aligning a plurality of protein or nucleic acid sequences comprising the steps of:
a) performing an alignment of a query sequence to a target sequence using a dynamic programming algorithm that constructs the alignment using a scoring matrix profile to provide an alignment score for aligning amino acid residues together, wherein suitable candidate residues for alignment are given a positive score and unsuitable candidate residues are given a negative score, and negative score penalties are generated both for opening and for extending a gap in one of the sequences in the alignment; and
b) repeating step a) for each sequence to be aligned; wherein the scoring matrix profile is modified after each alignment step a) and before being used to generate the alignment of the next sequence, and wherein if the best scoring alignment requires that a gap be introduced into the profile, the profile is modified by inserting the residues from the query sequence that match up with the gap region.

2. A method according to claim 1, wherein if amino acid residues or nucleotides in a second or subsequent query sequence are aligned against a modified region of the profile where residues or nucleotides have been inserted and said amino acid residues or nucleotides are assigned a negative score, their score is reset to zero, such that multiple sequences that have similar regions that were not present in the original profile may be aligned together without penalty while at the same time allowing the alignment score to be increased for correctly aligned regions that have a positive score.

3. A method according to either claim 1 or claim 2, wherein if the alignment of a second or subsequent query sequence requires that a gap be inserted or extended into the sequence that is being aligned against the profile and this gap falls within a modified region of the profile where residues or nucleotides have been inserted, no negative score penalty is generated, such that sequence that would normally align against the profile without the need for a gap can be aligned without an inserted region interfering with the alignment.

4. A method according to any one of the preceding claims, wherein if a query sequence is known to align against a target sequence in multiple locations such that multiple alignment hits are generated by the alignment of these sequences, then step a) is repeated for each location at which the sequences align, and for each separate iteration, the alignment of the sequences is constrained to one particular alignment location.

5. A method according to claim 4, wherein the alignment is constrained by excluding regions from consideration by the dynamic programming algorithm by setting the matrix profile scores in the excluded region to a large negative value beyond a value that would occur naturally during the execution of the algorithm.

6. A method according to claim 5, wherein the large negative value assigned is the largest negative value that can be stored by the computer on which the alignment method is being performed.

7. A method according to any one of the preceding claims, wherein the scoring matrix profile that is used in the alignment method is a profile generated by running a profile-based alignment algorithm on the target sequence.

8. A method according to claim 7, wherein the profile-based alignment algorithm is the position specific iterated basic local alignment search tool (PSI-BLAST).

9. A method according to any one of claims 1-7, wherein the scoring matrix profile that is used in the alignment method is a default scoring matrix.

10. A method according to claim 9, wherein said default matrix is a BLOSUM or PAM matrix.

11. A computer apparatus adapted to perform a method according to any one of the preceding claims.

12. A computer apparatus according to claim 11 comprising:
a processor means comprising:
a memory means adapted for storing data relating to amino acid or nucleotide sequences;
means for inputting data relating to a plurality of protein or nucleic acid sequences;
computer software means stored in said computer memory adapted to align said plurality of protein or nucleic acid sequences and output a multiple alignment of said sequences.

13. A computer-based system for aligning a plurality of protein or nucleic acid sequences comprising:
means for inputting data relating to a plurality of protein or nucleic acid sequences;
means adapted to align said plurality of protein or nucleic acid sequences; and
means for outputting a multiple alignment of said sequences.

14. A system according to claim 13, wherein said means adapted to align said plurality of protein or nucleic acid sequences is a computer software means.

15. A system according to either of claims 13 or 14, comprising:
a central processing unit;
an input device for inputting requests;
an output device;
a memory;
at least one bus connecting the central processing unit, the memory, the input device and the output device;
the memory storing a module that is configured so that upon receiving a request to align a plurality of protein or nucleic acid sequences, it performs the steps listed in any one of claims 1-10.

16. A computer program product for use in conjunction with a computer, said computer program comprising a computer readable storage medium and a computer program mechanism embedded therein, the computer program mechanism comprising a module that is configured so that upon receiving a request to align a plurality of protein or nucleic acid sequences, it performs the steps listed in any one of claims 1-10.
